(19) United States
(12) Patent Application Publication
(10) Pub. No.: US 2023/0251179 A1
(43) Pub. Date: Aug. 10, 2023

(54) TURBIDITY MONITORING APPARATUS

(71) Applicant: THE WAVE TALK, INC., Daejeon (KR)

(72) Inventors: Young Dug KIM, Seongnam-si Gyeonggi-do (KR); Kyoung Man CHO, Seoul (KR)

(73) Assignee: THE WAVE TALK, INC., Daejeon (KR)

(21) Appl. No.: 18/115,109

(22) Filed: Feb. 28, 2023

Related U.S. Application Data
(63) Continuation of application No. PCT/KR2022/020675, filed on Dec. 19, 2022.

(30) Foreign Application Priority Data
Feb. 9, 2022 (KR) 10-2022-0017112

(51) Int. Cl. G01N 15/06

(52) U.S. Cl. CPC G01N 15/06; G01N 2015/0693; G01N 2015/0053

(57) ABSTRACT

Provided is a turbidity monitoring apparatus including a multi-tubular structure including an inner tube through a fluid to be measured flows and an outer tube that surrounds the inner tube, a wave source configured to irradiate a wave towards the multi-tubular structure, a detection unit configured to detect a laser speckle at each preset time, the laser speckle being generated by multiple scattering of the irradiated wave in the multi-tubular structure, and a control unit configured to estimate, in real time, the concentration of suspended substances or turbidity substances in the fluid to be measured, by using the detected laser speckles.

### CROSS-REFERENCE TO RELATED APPLICATION

The present application is a continuation of International Application No. PCT/KR2022/020675, filed on Dec. 19, 2022, which claims the benefit of Korean Patent Application No. 10-2022-0017112 filed on Feb. 9, 2022 in the Korean Intellectual Property Office, the entire disclosure of each of which is incorporated herein by reference.

### BACKGROUND

### 1. Field

Embodiments of the present disclosure relate to a turbidity monitoring apparatus.

### 2. Description of the Related Art

Turbidity is a quantitative measure of cloudiness in water and refers to the resistance to light transmission. Turbidity is caused by a variety of suspended solids, and the size of turbidity particles varies from colloidal dispersion to coarse dispersion. Substances that cause turbidity are very diverse, ranging from pure inorganic substances to mainly natural organic substances, and specifically, from pure inorganic substances, such as earth flows, to natural organic substances, or bacteria, microorganisms, algae and the like generated by large amounts of inorganic substances and organic substances introduced from factory wastewater and domestic sewage also act as turbidity-inducing substances.

A turbidity measuring apparatus is an essential element in a water quality measurement system for water supply and drainage, and requires a wide range of turbidity measurements according to water quality characteristics (feed water, precipitated water, purified water, flushed water, and the like). Turbidity measuring apparatuses for measuring the quality of tap water can be divided into high-concentration turbidity meters for measuring high-concentration turbidity, such as feed tap water and flushed effluent water, and low-concentration turbidity meters for measuring low-concentration turbidity, such as purified tap water.

Conventionally, turbidity can be monitored by measuring the turbidity of a continuously supplied fluid, that is, water, by using such a turbidity measuring apparatus. However, a biofilm is formed by microorganisms such as bacteria in a pipe through which a fluid flows, and thus, it is difficult to accurately measure turbidity unless a turbidity measuring apparatus is regularly maintained and managed.

### SUMMARY

One or more embodiments provide a turbidity monitoring apparatus capable of measuring high-concentration samples and minimizing regular maintenance and management.

Additional aspects will be set forth in part in the description which follows and, in part, will be apparent from the description, or may be learned by practice of the presented embodiments of the disclosure.

An embodiment of the present disclosure provides a turbidity monitoring apparatus including a multi-tubular structure including an inner tube through which a fluid to be measured flows and an outer tube that surrounds the inner tube, a wave source configured to irradiate a wave towards the multi-tubular structure, a detection unit configured to detect a laser speckle generated by multiple scattering of the irradiated wave inside the multi-tubular structure, at each preset time, and a control unit configured to estimate, in real time, a concentration of suspended substances or turbidity substances in the fluid to be measured, by using the detected laser speckles.

In one embodiment of the present disclosure, at least a portion of the inner tube of the multi-tubular structure may be made of a light-transmitting material.

In one embodiment of the present disclosure, the outer tube of the multi-tubular structure may include a multiple scattering amplifier for amplifying the number of multiple scatterings of the wave irradiated from the wave source inside the inner tube.

In one embodiment of the present disclosure, the control unit may determine a dilution factor of suspended substances or turbidity substances in the fluid to be measured, by using a first diameter of the inner tube and a second diameter of the outer tube, and estimate a concentration of the suspended substances or the turbidity substances on the basis of the dilution factor.

In one embodiment of the present disclosure, the inner tube of the multi-tubular structure may be co-axial with the outer tube of the multi-tubular structure.

In one embodiment of the present disclosure, a first central axis of the inner tube of the multi-tubular structure may be parallel to a second central axis of the outer tube of the multi-tubular structure.

Other aspects, features and advantages other than those described above will become apparent from the following drawings, claims and detailed description of the disclosure.

### BRIEF DESCRIPTION OF THE DRAWINGS

The above and other aspects, features, and advantages of certain embodiments of the disclosure will be more apparent from the following description taken in conjunction with the accompanying drawings, in which:

FIG. 1 is a schematic conceptual diagram illustrating a turbidity monitoring apparatus according to an embodiment of the present disclosure;

FIG. 2 is a diagram for explaining the measurement principle of a turbidity monitoring apparatus according to an embodiment of the present disclosure;

FIG. 3 is a conceptual view for explaining a multi-tubular structure, according to an embodiment of the present disclosure;

FIGS. 4A to 6B are views illustrating various embodiments of the multi-tubular structure of FIG. 3;

FIG. 7 is a schematic conceptual diagram for explaining a turbidity monitoring apparatus according to another embodiment of the present disclosure; and

FIG. 8 is a block diagram of the turbidity monitoring apparatus of FIG. 7.

### DETAILED DESCRIPTION

Reference will now be made in detail to embodiments, examples of which are illustrated in the accompanying drawings, wherein like reference numerals refer to like elements throughout. In this regard, the present embodiments may have different forms and should not be construed as being limited to the descriptions set forth herein. Accordingly, the embodiments are merely described below, by referring to the figures, to explain aspects. As used herein, the term “and/or” includes any and all combinations of one or more of the associated listed items. Expressions such as “at least one of,” when preceding a list of elements, modify the entire list of elements and do not modify the individual elements of the list.

Hereinafter, the following embodiments will be described in detail with reference to the accompanying drawings, and when describing with reference to the drawings, the same or corresponding components are given the same reference numerals, and redundant description thereof will be omitted.

The present embodiments allow for various changes, and specific embodiments will be illustrated in the drawings and described in detail in the detailed description. Effects and characteristics of the present embodiments, and methods for achieving them will become apparent from the details described below with reference to the drawings. However, the present embodiments are not limited to the embodiments disclosed below and may be implemented in various forms.

In the following embodiments, terms such as “first” and “second” are used for the purpose of distinguishing one component from another component not for purposes of limitation.

In the following embodiments, singular expressions include plural expressions unless the context clearly indicates otherwise.

In the following embodiments, terms such as “include” or “have” mean that features or elements described in the specification are present, and do not preclude the possibility that one or more other features or elements may be added.

In the following embodiments, when a portion such as a unit, a region, or a component is referred to as being above or on another portion, this includes not only the case of being directly above the other portion, but also the case where another unit, region or component may be present therebetween.

In the following embodiments, terms such as “connected” or “coupled” do not necessarily mean direct and/or fixed connection or coupling of two members unless the context clearly indicates otherwise, and do not preclude that another member is interposed between the two members.

It means that the features or components described in the specification exist, and does not preclude the possibility that one or more other features or components may be added.

In the drawings, the size of components may be exaggerated or reduced for convenience of explanation. For example, the size and thickness of each component shown in the drawings are arbitrarily illustrated for convenience of explanation, and thus, the following embodiments are not necessarily limited thereto.

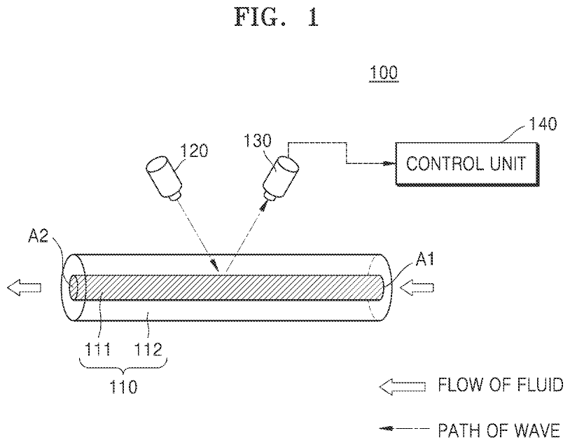

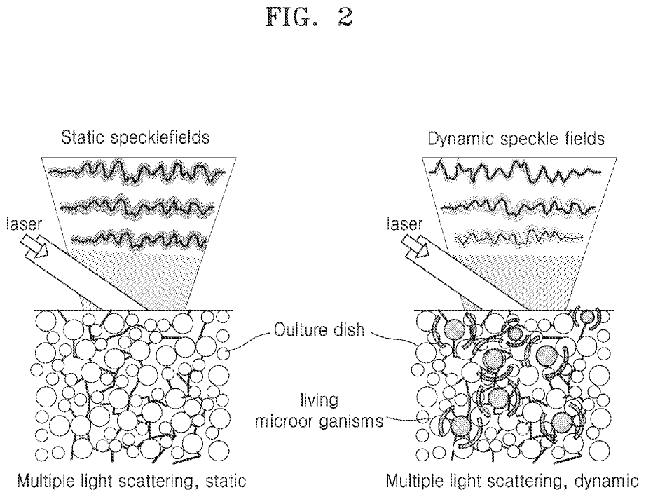

FIG. 1 is a schematic conceptual diagram illustrating a turbidity monitoring apparatus 100 according to an embodiment of the present disclosure, and FIG. 2 is a view for explaining the measurement principle of a turbidity monitoring apparatus according to an embodiment of the present disclosure.

Referring to FIG. 1, the turbidity monitoring apparatus 100 according to an embodiment of the present disclosure may include a multi-tubular structure 110, a wave source 120, a detection unit 130, and a control unit 140.

The multi-tubular structure 110 may include an inner tube 111 through which a fluid to be measured flows, and an outer tube 112 that surrounds the inner tube 111. In the multi-tubular structure 110, a fluid introduced through a first cross-section A1 of the inner tube 111 may be discharged through a second cross-section A2 of the inner tube 111.

In this regard, the fluid may be liquid or gas. In addition, the fluid may include a material in which microorganisms can grow, and may be, for example, water such as tap water or sewage. The fluid may include suspended substances having a particle diameter of 2 μm or more that are insoluble in water, or turbidity substances having a particle diameter of less than 2 μm.

As the amount of the suspended substances or the turbidity substances in the fluid is larger, it is more difficult to distinguish the difference in concentration in the fluid. To solve this obstacle, the multi-tubular structure 110 according to one embodiment of the present disclosure includes the inner tube 111 through which a fluid flows and is configured to accurately measure the turbidity in a high-concentration fluid by using the relationship between the inner tube 111 and the outer tube 112 that surrounds the inner tube 111.

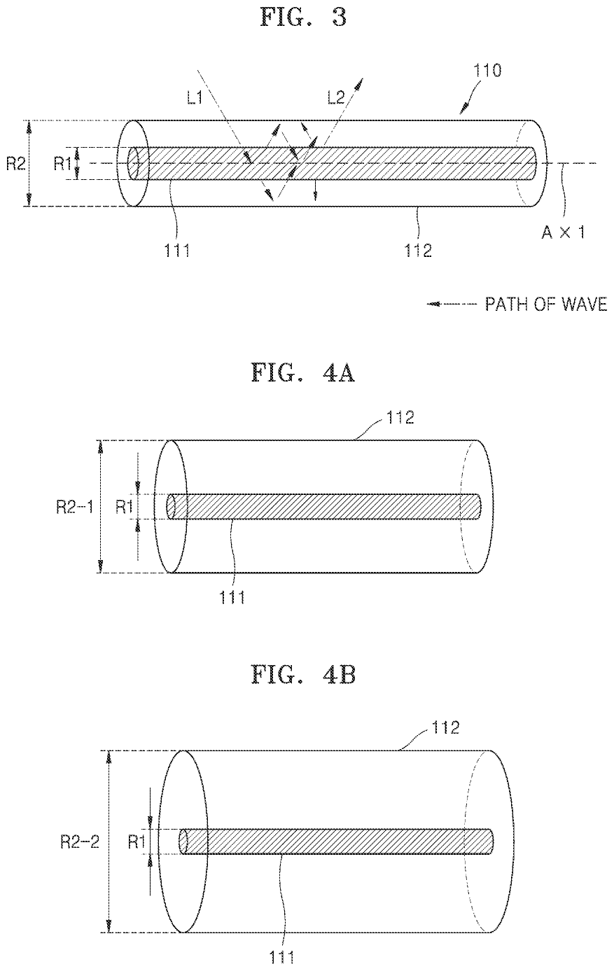

The multi-tubular structure 110 may constitute at least a portion of a water supply system or a sewage system. The multi-tubular structure 110 may be arranged at one or more locations for monitoring water quality, turbidity, or the like in a water supply system or a sewage system. The multi-tubular structure 110 will be described in more detail with reference to FIGS. 3 to 6B.

The wave source 120 may irradiate a wave having coherence towards the multi-tubular structure 110. In this regard, any types of source devices capable of generating waves may be applied as the wave source 120, and the wave source 120 may be a laser capable of irradiating light of a specific wavelength band.

In this regard, a laser having good coherence may be used as the wave source 120, to form a speckle, which is an interference pattern in the fluid flowing through the inner tube 111. At this time, the shorter the spectral bandwidth of a light source that determines the coherence of the laser light source, the higher the measurement accuracy.

That is, the longer the coherence length, the higher the measurement accuracy. Accordingly, laser light having a spectral bandwidth less than a predefined reference bandwidth of the wave source 120 may be used as the wave source 120, and the shorter the reference bandwidth, the higher the measurement accuracy. For example, the spectral bandwidth of the light source may be set so that the condition of Expression 1 below is maintained.

Spectral bandwidth<5 nm [Expression 1]

According to Expression 1, to measure the pattern change of a laser speckle, the spectral bandwidth of the wave source 120 may be maintained at less than 5 nm when the multi-tubular structure 110 is irradiated with light.

The detection unit 130 may detect a laser speckle generated when the irradiated wave is multi-scattered inside the multi-tubular structure 110, at each preset time. The detection unit 130 may be arranged on the multi-tubular structure 110. In particular, the detection unit 130 may be arranged on the multi-tubular structure 110 between the first cross-section A1 and the second cross-section A2 of the inner tube 111. The detection unit 130 may be a CCD camera. The detection unit 130 may measure an optical image emitted from the multi-tubular structure 110 and provide the measured optical image to the control unit 140.

The term “time” as used herein refers to any moment in the continuous flow of time, and times may be preset at the same time interval, but is not necessarily limited thereto, and may also be preset at certain time intervals.

For example, in the case of using a light source in the visible light wavelength range is used, a CCD camera, which is an image pickup device for capturing an image, may be used. The detection unit 130 may detect at least a laser speckle at a first time and a laser speckle at a second time, and provide the detected laser speckles to the control unit 140. Meanwhile, the first time and the second time are only examples selected for convenience of explanation, and the detection unit 130 may detect laser speckles at a plurality of times greater than the first time and the second time.

Hereinafter, the principle of monitoring turbidity, according to the present disclosure, will be described.

When light is irradiated to a material having a uniform internal refractive index, e.g., glass, the light is refracted in a certain direction. However, when coherent light such as a laser is irradiated to a material having a non-uniform internal refractive index, very complicated multiple scattering occurs in the material.

Referring to FIG. 2, in light or waves (hereinafter, referred to as waves for simplicity) irradiated from a wave source, some of the waves scattered through complicated paths due to the multiple scattering passes through a surface to be inspected. Waves passing through multiple points in the surface to be inspected cause constructive interference or destructive interference, and the constructive/destructive interference of the waves generates a grain-shaped pattern (speckles).

In the present specification, the waves scattered in the complicated paths are referred to as “chaotic wave,” and the chaotic wave may be detected through laser speckles.

The left side of FIG. 2 shows a state in which a laser is irradiated to a stable medium. When a stable medium, in which an internal constituent material does not move, is irradiated with interference light (e.g., laser), a stable speckle pattern without a variation may be observed.

However, as illustrated in the right side of FIG. 2, when an unstable medium, in which an internal constituent material, such as bacteria, is moving, is included inside, the speckle pattern changes.

That is, due to microscopic biological activities of microorganisms (e.g., intracellular movement, movement of microorganisms, and movement of mites) or movement of minute turbidity substances in a fluid, a fine change in the optical path may occur over time. The speckle pattern is a phenomenon generated by interference of waves, and thus, a fine change in the optical path may cause variation in the speckle pattern. Accordingly, when a temporal variation in the speckle pattern is measured, the movement of living organisms or the movement of minute turbidity substances in a fluid may be rapidly measured. As such, when the variation in the speckle pattern over time is measured, the presence or absence of living organisms and the concentration of turbidity substances may be identified, and further, types of living organisms may also be identified.

In the present specification, a configuration for measuring the variation in the speckle pattern is defined as a chaotic wave sensor.

Referring back to FIGS. 1 and 2, when a wave is irradiated to a fluid in the multi-tubular structure 110, the incident wave may form a laser speckle by multiple scattering in the fluid. Since the laser speckle is generated by the interference of light, if turbidity substances are constant in the fluid, a constant interference pattern may always be shown over time.

In contrast, when turbidity substances change in the fluid, the laser speckle may change over time by a change of the turbidity substances. The detection unit 130 may detect a laser speckle that changes over time at each preset time and may provide the detected laser speckles to the control unit 140.

The detection unit 130 needs to be capable of high-speed measurement to measure turbidity from a flowing fluid. The term “high-speed measurement” as used herein refers to the detection of a laser speckle faster than the flow rate of a fluid. For example, the measurement speed of the detection unit 130 may be set faster than the rate of the fluid flowing in the multi-tubular structure 110.

Meanwhile, when an image sensor is used as the detection unit 130, the image sensor may be arranged so that a size d of one pixel of the image sensor is equal to or less than the grain size of the speckle pattern. For example, an image sensor may be arranged in an optical system included in the detection unit 130 to satisfy the condition of Expression 2 below.

d≤speckle grain size [Expression 2]

As shown in Expression 2, the size d of one pixel of the image sensor needs to be equal to or less than the grain size of the speckle pattern. However, when the size of the pixel is too small, undersampling occurs, and thus, there may be a difficulty in using pixel resolution. Accordingly, to achieve an effective signal to noise ratio (SNR), the image sensor may be arranged such that a maximum of 5 or less pixels are located in a speckle grain size.

The control unit 140 may estimate, in real time, the concentration of suspended substances or turbidity substances in the fluid to be measured, by using the detected laser speckles. The control unit 140 may estimate, in real time, the concentration of suspended substances or turbidity substances in the fluid on the basis of the obtained temporal correlation. The term “real-time” as used herein refers to estimating the concentration within 3 seconds, and preferably refers to estimating the concentration within 1 second.

In one embodiment, the control unit 140 may estimate the concentration of suspended substances or turbidity substances in the fluid by using a difference between first image information of a laser speckle detected at a first time, and second image information of a second laser speckle detected at a second time different from the first time.

In this regard, the first image information and the second image information may be at least one of laser speckle pattern information and wave intensity information. Meanwhile, in one embodiment of the present disclosure, only the difference between the first image information at the first time and the second image information at the second time is not used, and further, a plurality of pieces of laser speckle image information may be used at a plurality of times.

The control unit 140 may calculate a temporal correlation coefficient between images by using image information of laser speckles generated at a plurality of preset times, and may estimate the concentration of suspended substances or turbidity substances in the fluid on the basis of the temporal correlation coefficient. The temporal correlation between the detected laser speckle images may be calculated using Expression 3 below. However, Expression 3 below is only an example, and the temporal correlation may also be derived using other expressions.

$$\overline{C} ( x , y ; τ ) = \frac{1}{T - τ} {\sum }_{t = 1}^{T - τ} \overline{I} ( x , y ; t ) \overline{I} ( x , y ; t + τ ) δ t \qquad [ Expression 3 ]$$

wherein, in Expression 3, C̅ refers to a temporal correlation coefficient, Ī refers to normalized light intensity, (x,y) refers to pixel coordinates of a camera, t refers to measurement time, T refers to total measurement time, τ refers to a time lag.

The temporal correlation coefficient may be calculated according to Expression 3, and in one embodiment, the concentration of suspended substances or turbidity substances in the fluid may be estimated through an analysis in which the temporal correlation coefficient falls below a preset reference value. In addition, the control unit 140 may estimate the concentration of suspended substances or turbidity substances in the fluid by using a change rate or peak value of the temporal correlation coefficient.

In another embodiment, the control unit 140 may obtain a spatial correlation of the interference pattern. In this regard, the spatial correlation given by the following expression below may be as follows: How similar the brightness of a pixel and the brightness of a pixel spaced apart from the pixel by a distance r, on an image measured at a time t, are may be expressed as a value within a certain range. The certain range may be between −1 and 1. That is, the spatial correlation indicates the degree of correlation between a pixel and another pixel. 1 denotes a positive correlation, −1 denotes a negative correlation, and 0 denotes no correlation. Specifically, the brightness is uniform before the interference pattern is formed, and thus, the spatial correlation of a sample image shows a positive correlation close to 1, but the value of the correlation may fall in a direction close to 0 after the interference pattern is formed.

In the detection unit 130, the brightness of a pixel at a position r′=(x,y), measured at a time t may be defined as I(r′,t), and the brightness of a pixel spaced apart therefrom by a distance r may be defined as I(r′+r, t). The spatial correlation defined using the same may be expressed by Expression 4 below.

$$C ( r , t ) = \frac{1}{{C}_{0} ( t )} \int \int I ( {r}^{′} + r , t ) I ( {r}^{′} , t ) {dr}^{′} \qquad [ Expression 4 ]$$

C₀(t) was used to set the range of Expression 4 to between −1 and 1. When the brightness I(r′,t) of a pixel, measured at a time t and the brightness I(r′+r,t) of a pixel spaced apart therefrom by a distance r are the same, the spatial correlation is derived as 1, and, if not, the spatial correlation has a value less than 1.

In one embodiment of the present disclosure, the spatial correlation may also be expressed only by a function of time. To this end, the control unit 140 may calculate an average of spatial correlations between pixels spaced apart from a pixel by the same distance r by Expression 5 below.

$$C ( ρ , t ) = \frac{1}{2 π} {\int }_{0}^{2 π} C ( r , t ) d θ \qquad [ Expression 5 ]$$

In one embodiment, the control unit 140 may express Equation 5 as a function of time by substituting a preset distance into Equation 5, and this function may be used to determine the degree of formation of an interference pattern as a value within a certain range of 0 to 1.

The control unit 140 may determine the concentration information of suspended substances or turbidity substances by using the spatial correlation as follows. The spatial correlation may be obtained by creating two identical images superimposed using one image, shifting one of the two images in one direction by a present distance, and then analyzing how similar two pixels are between the shifted image and the non-shifted image. In this regard, the spatial correlation is a measure of how uniform an image is. When an interference pattern is formed by suspended substances or turbidity substances, the similarity between two adjacent pixels is reduced due to a small interference pattern, and thus, a value of the spatial correlation also falls.

The spatial correlation coefficient varies according to the shifted distance r. As the shifting distance r increases within a certain distance range, the spatial correlation coefficient decreases, and, when the shifted distance r exceeds the certain distance range, the spatial correlation coefficient has an almost constant value. Accordingly, to obtain a more meaningful spatial correlation, the control unit 140 may obtain the spatial correlation by shifting an image by a certain preset distance or more. In this regard, the preset certain distance r depends on the speckle size, and the control unit 140 may obtain the spatial correlation by shifting an image by a pixel that is greater than the speckle size when expressed in units of pixels.

Meanwhile, the control unit 140 may obtain a temporal correlation of the interference pattern of a measured sample image, as well as the above-described spatial correlation, and may detect the concentration of suspended substances or turbidity substances on the basis of the obtained temporal correlation. The control unit 140 may calculate a temporal correlation coefficient between images by using image information of the interference pattern measured in a time-serial manner, and may estimate the concentration of suspended substances or turbidity substances in the fluid on the basis of the temporal correlation coefficient.

Hereinafter, the multi-tubular structure 110 will be described in detail with reference to the drawings.

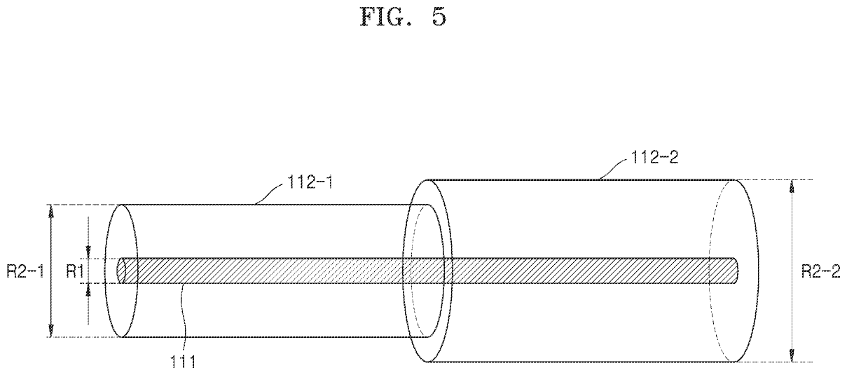

FIG. 3 is a conceptual view for explaining the multi-tubular structure 110, according to an embodiment of the present disclosure, and FIGS. 4A to 6B are views illustrating various embodiments of the multi-tubular structure 110 of FIG. 3.

First, referring to FIG. 3, the multi-tubular structure 110 may be made of a double tube including the inner tube 111 and the outer tube 112 that surrounds the inner tube 111. At least a portion of the inner tube 111 may be made of a light-transmitting material. The inner tube 111 may have a tubular shape in which a fluid can flow in through a first cross-section A1 and be discharged through a second cross-section A2.

The outer tube 112 may be made of the same material as the inner tube 111, and may also be made of a material different from the inner tube 111. The outer tube 112 may be made of a light-transmitting material, and may have a tubular shape with the inner tube 111 arranged therein. When the outer tube 112 has a tubular shape, a support member (not shown) for supporting the gap between the outer tube 112 and the inner tube 111 may be further included, to maintain a tubular shape between the outer tube 112 and the inner tube 111.

In another embodiment, in the multi-tubular structure 110, the gap between the inner tube 111 and the outer tube 112 may also be filled with a light-transmitting material. In this regard, the material that fills the gap between the inner tube 111 and the outer tube 112 may be the same as the materials of the inner tube 111 and the outer tube 112. In this case, the multi-tubular structure 110 may be a structure with an inside that is hollow as much as a first diameter R1 of the inner tube 111. A multiple scattering amplifying material capable of amplifying multiple scattering may further fill the gap between the inner tube 111 and the outer tube 112. For example, the multiple scattering amplifying material may include particles having a high refractive index and a diameter of a micrometer size or less, for example, titanium oxide (TiO₂) nanoparticles.

In another embodiment, the multi-tubular structure 110 may further include a multiple scattering amplification region in the inner tube 111 or the outer tube 112. The multiple scattering amplification region may be formed by coating on the inner tube 111 or the outer tube 112, and a pattern for amplifying multiple scattering may also be formed on an inner surface of the inner tube 111 or an inner surface of the outer tube 112.

When a first wave L1 is incident on the multi-tubular structure 110 from the wave source 120, the first wave L1 may be irradiated to the inner tube 111 via the outer tube 112, and may be scattered in a fluid flowing through the inner tube 111. The scattered waves cause constructive interference or destructive interference, and the constructive/destructive interference of the waves may be detected by the detection unit 130 as a second wave L2 emitted after generating a bullet-shaped pattern (speckle).

In this case, the control unit 140 may determine the dilution factor of suspended substances or turbidity substances in the fluid to be measured, by using the first diameter R1 of the inner tube 111 and a second diameter R2 of the outer tube 112. Specifically, a fluid flows only through the inner tube 111, and the waves scattered in the inner tube 111 may be scattered again in the outer tube 112, thereby reducing the degree of scattering. In other words, the turbidity monitoring apparatus 100 may determine the resolution detected according to a diameter ratio of the outer tube 112 to the inner tube 111.

For example, when comparing FIGS. 4A and 4B, in the case in which a second diameter R2-1 of the outer tube 112 is different from a second diameter R2-2 of the outer tube 112 even though the first diameters R1 of the inner tubes 111 are the same, the dilution factor may vary. In other words, the second diameter R2-2 of the outer tube 112 in FIG. 4B is larger than the second diameter R2-1 of the outer tube 112 in FIG. 4A, and thus, the dilution factor of the multi-tubular structure 110 in FIG. 4B may be larger.

According to the present disclosure, the control unit 140 estimates the concentration of suspended substances or turbidity substances in the fluid by using the temporal correlation or the spatial correlation of laser speckles formed by scattering in the fluid, and, when a high-concentration fluid is an object, the degree of scattering is large, and thus, it may be difficult to detect each concentration separately. To this end, in the present disclosure, in the case in which a high-concentration fluid needs to be measured, through dilution using the structure of the multi-tubular structure 110, the concentration of suspended substances or turbidity substances in the fluid may be accurately distinguished and detected.

The control unit 140 determines the dilution factor of suspended substances or turbidity substances in the fluid to be measured, by using the first diameter R1 of the inner tube 111 and the second diameter R2 of the outer tube 112, and then may estimate the concentration of the suspended substances or the turbidity substances by using the dilution factor.

In another embodiment, as illustrated in FIG. 5, the multi-tubular structure 110 may include a first outer tube 112-1 and a second outer tube 112-2 that have different diameters. In this case, the first diameters R1 of the inner tubes 111 may be the same, but the present disclosure is not necessarily limited thereto, and the multi-tubular structure 110 may include an inner tube 111 having a different diameter, like the first outer tube 112-1 and the second outer tube 112-2.

Meanwhile, in one embodiment, as illustrated in FIG. 3, the inner tube 111 of the multi-tubular structure 110 may be coaxial with the outer tube 112 of the multi-tubular structure 10. However, the present disclosure is not limited thereto, and, as illustrated in FIGS. 6A and 6B, a first central axis Ax1 of the inner tube 111 of the multi-tubular structure 110 may be parallel to a second central axis Ax2 of the outer tube 112 of the multi-tubular structure 110.

As illustrated in FIG. 6A, the inner tube 111 may be arranged at a position spaced apart from the second central axis Ax2 of the outer tube 112. In this case, one or more detection units 130 arranged outside may be provided to detect laser speckles emitted at different positions. Thus, the turbidity monitoring apparatus 100 can more accurately and rapidly detect turbidity.

As illustrated in FIG. 6B, the multi-tubular structure 110 may include two or more inner tubes (111). When the multi-tubular structure 110 includes two inner tubes, i.e., a first inner tube 111-1 and a second inner tube 111-2, a 1-1 central axis Ax1-1 of the first inner tube 111-1 and a 1-2 central axis Ax1-2 of the second inner tube 111-2 may be parallel to the second central axis Ax2 of the outer tube 112. When the multi-tubular structure 110 includes two or more inner tubes, i.e., the first inner tube 111-1 and the second inner tube 111-2, turbidity may be detected while the same fluid is allowed to flow through the first inner tube 111-1 and the second inner tube 111-2, which are different, or turbidity may be detected while different fluids are allowed to flow through the first inner tube 111-1 and the second inner tube 111-2.

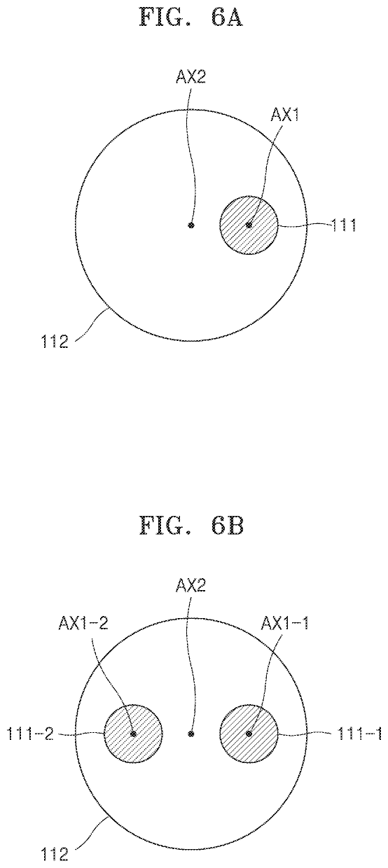

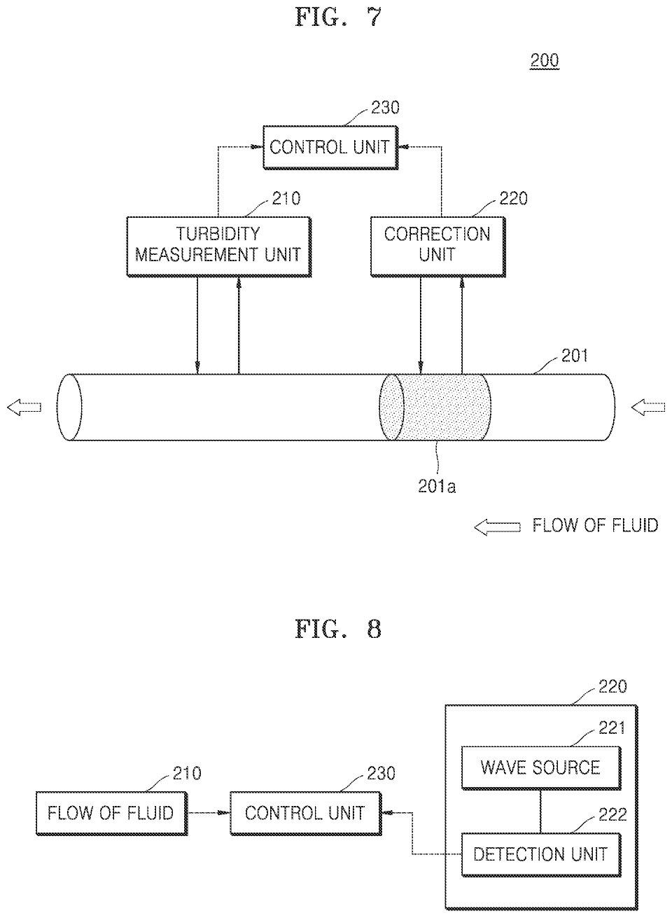

FIG. 7 is a schematic conceptual diagram for explaining a turbidity monitoring apparatus 200 according to another embodiment of the present disclosure, and FIG. 8 is a block diagram of the turbidity monitoring apparatus 200 of FIG. 7.

Referring to FIGS. 7 and 8, the turbidity monitoring apparatus 200 according to another embodiment of the present disclosure may include a turbidity measurement unit 210, a correction unit 220, and a control unit 230. The turbidity monitoring apparatus 200 according to another embodiment of the present disclosure measures the turbidity of a fluid accommodated in an accommodation unit 201 through a conventional turbidity measurement unit 210. In this case, a measured value is corrected by the correction unit 220 using a chaotic wave sensor, to accurately measure the turbidity. Although the drawings illustrate the accommodation unit 201 as having a pipe shape, the present disclosure is not limited thereto, and the accommodation unit 201 may also have any shape that can be applied to the conventional turbidity measurement unit 210.

The turbidity measurement unit 210 is a device for quantitatively displaying cloudiness in water, and may be a device used to measure water quality together with a pH meter, a biochemical oxygen demand (BOD), a conductivity meter, and the like. In the present disclosure, there is no limitation on the turbidity measurement unit 210, and any commercially available products, devices, or the like may be applied.

The correction unit 220 may include a wave source 221 configured to irradiate a wave to the accommodation unit 201 and a detection unit 222 configured to detect a multi-scattered laser speckle emitted from the accommodation unit 201. The wave source 221 and the detection unit 222 have the same configuration as the wave source 120 and the detection unit 130 as described above, and thus, redundant descriptions will be omitted for convenience of explanation.

The wave source 221 may emit a wave having coherence towards the accommodation unit 201. In this case, any types of source devices capable of generating waves may be applied as the wave source 221, and the wave source 221 may be a laser capable of irradiating light of a specific wavelength band.

In this case, the accommodation unit 201 may further include a multiple scattering amplification region 201a for further amplifying the multiple scattering of light emitted from a fluid in the accommodation unit 201. For example, the multiple scattering amplification region 201a may be formed by coating on the accommodation unit 201.

The detection unit 222 may detect a laser speckle, which is generated by multiple scattering of the irradiated wave in the accommodation unit 201, at each preset time. The detection unit 222 may be arranged on the accommodation unit 201.

The control unit 230 may estimate, in real time, the concentration of suspended substances or turbidity substances in the fluid to be measured, by using laser speckles detected from the accommodation unit 201. In this case, the control unit 230 may receive first measurement data from the turbidity measurement unit 210 and receive second measurement data from the correction unit 220. The control unit 230 may estimate the concentration of suspended substances or turbidity substances in the fluid on the basis of the first measurement data, and may correct the value by using the second measurement data.

When the accommodation unit 201 continues to accommodate a fluid, a biofilm may be formed inside the accommodation unit 201 by bacteria or the like in the fluid. In this case, it is difficult for the turbidity measurement unit 210 to perform accurate measurement due to the biofilm, which requires more regular maintenance and management.

The turbidity monitoring apparatus 200 according to the present disclosure may accurately extract second measurement data related to the turbidity of the fluid in the same accommodation unit 201 even through a biofilm is formed, through the correction unit 220 for measuring a change in the laser speckle over time. Thus, the turbidity monitoring apparatus 200 does not directly detect turbidity through the correction unit 220, but obtains reference data, and, by correcting the first measurement data of the turbidity measurement unit 210 on the basis thereof, may accurately measure suspended substances or turbidity substances in the fluid.

As described above, the turbidity monitoring apparatus according to embodiments of the present disclosure can realize the effect of diluting a high-concentration fluid by using a multi-tubular structure, and thus can accurately measure suspended substances or turbidity substances in the high-concentration fluid.

In addition, the turbidity monitoring apparatus according to embodiments of the present disclosure can correct the measurement result of an existing turbidity measurement unit by acquiring turbidity-related data using a change in a laser speckle image over time, and thus requires less regular maintenance and management, and can increase the accuracy of detecting turbidity in a fluid.

Exemplary embodiments of the present disclosure have been described. It will be understood by those of ordinary skill in the art to which the present disclosure pertains that the present disclosure may be carried out in modified forms without departing from the essential characteristics of the present disclosure. Therefore, embodiments disclosed herein should be considered in a descriptive sense only and not for purposes of limitation. The scope of the present disclosure is shown in the claims rather than the foregoing description, and all differences within the equivalent scope should be construed as being included in the present disclosure.

It should be understood that embodiments described herein should be considered in a descriptive sense only and not for purposes of limitation. Descriptions of features or aspects within each embodiment should typically be considered as available for other similar features or aspects in other embodiments. While one or more embodiments have been described with reference to the figures, it will be understood by those of ordinary skill in the art that various changes in form and details may be made therein without departing from the spirit and scope as defined by the following claims.

What is claimed is:

1. A turbidity monitoring apparatus comprising:
a multi-tubular structure including
an inner tube through which a fluid to be measured flows and
an outer tube that surrounds the inner tube;
a wave source configured to irradiate a wave towards the multi-tubular structure;
a detection unit configured to detect a laser speckle at each preset time, the laser speckle being generated by multiple scattering of the irradiated wave in the multi-tubular structure; and
a control unit configured to estimate, in real time, a concentration of suspended substances or turbidity substances in the fluid to be measured, by using the detected laser speckles.

2. The turbidity monitoring apparatus of claim 1, wherein at least a portion of the inner tube of the multi-tubular structure includes a light-transmitting material.

3. The turbidity monitoring apparatus of claim 1, wherein the outer tube of the multi-tubular structure comprises a multiple scattering amplifier configured to amplify a number of multiple scatterings of the wave irradiated from the wave source in the inner tube.

4. The turbidity monitoring apparatus of claim 1, wherein the control unit is configured to determine a dilution factor of suspended substances or turbidity substances in the fluid to be measured, by using a first diameter of the inner tube and a second diameter of the outer tube, and estimate a concentration of the suspended substances or turbidity substances on the basis of the dilution factor.

5. The turbidity monitoring apparatus of claim 1, wherein the inner tube of the multi-tubular structure is coaxial with the outer tube of the multi-tubular structure.

6. The turbidity monitoring apparatus of claim 1, wherein a first central axis of the inner tube of the multi-tubular structure is parallel to a second central axis of the outer tube of the multi-tubular structure.
